(19) United States
(12) Patent Application Publication
(10) Pub. No.: US 2003/0135069 A1
(43) Pub. Date: Jul. 17, 2003

(54) Catalyst for use in producing lower aliphatic carboxylic acid ester, process for producing the catalyst,and process for producing lower aliphatic carboxylic acid ester using the catalyst

(76) Inventors: Ayumu Fujita, Oita (JP); Etsuko Kadowaki, Oita (JP); Tomoyoshi Higashi, Oita (JP); Hiroshi Uchida, Oita (JP)

Correspondence Address: SUGHRUE MION, PLLC, 2100 PENNSYLVANIA AVENUE, N.W., WASHINGTON, DC, 20037, US

(21) Appl. No.: 09/958,096

(22) PCT Filed: Sep. 5, 2001
(86) PCT No.: PCT/JP01/07708

(30) Foreign Application Priority Data
Sep. 7, 2000 (JP) 2000-271415

(51) Int. Cl. C07C 69/02; B01J 27/19

(52) U.S. Cl. 560/231; 502/211

(57) ABSTRACT

A catalyst, for use in producing a lower aliphatic carboxylic acid ester, which is used in producing a lower aliphatic carboxylic acid ester by esterifying a lower aliphatic carboxylic acid with a lower alcohol in a gas phase and which can exhibit high initial activity and is able to stably and continuously perform the reaction. The catalyst comprises an inorganic support having supported thereon at least one heteropolyacid and/or a salt thereof.

### CROSS-REFERENCE TO RELATED APPLICATION

This application is an application filed under 35 U.S.C. §111(a) claiming benefit pursuant to 35 U.S.C. §119(e)(1) of the filing date of the Provisional Application No. 60/238,436 filed Oct. 10, 2000, pursuant to 35 §111(b).

### TECHNICAL FIELD

The present invention relates to a catalyst, for use in producing a lower aliphatic carboxylic acid ester, which is used in producing a lower aliphatic carboxylic acid ester from a lower alcohol and a lower aliphatic carboxylic acid in a gas phase; a process for producing the catalyst; and a process for producing a lower aliphatic carboxylic acid ester using the catalyst.

More specifically, the present invention relates to a catalyst for use in producing a lower aliphatic carboxylic acid ester, which comprises a heteropolyacid and/or a heteropolyacid salt and which is used in the process for producing a lower aliphatic carboxylic acid ester by esterifying a lower aliphatic carboxylic acid with a lower alcohol, wherein the catalyst is supported on a specific inorganic support; a process for producing the catalyst; and a process for producing a lower aliphatic carboxylic acid ester using the catalyst.

### BACKGROUND ART

Lower aliphatic carboxylic acid esters are widely used as various industrial starting materials or organic solvents and, for the production process thereof, various proposals have been made and implemented in industry.

Among the production processes, a process of producing a lower aliphatic carboxylic acid ester by esterifying a lower aliphatic carboxylic acid with a lower alcohol has long been known and particularly, in an area where the lower alcohol is inexpensively available, this production process has recently been noticed because of its merits in view of the starting material.

With respect to the production process starting from a lower aliphatic carboxylic acid and a lower alcohol, a process of obtaining a lower aliphatic carboxylic acid ester by a dehydration reaction using an acid catalyst is generally used and various studies are being made thereon. Specific examples thereof include those described in Japanese Examined Patent Publication No. 45-14529 (JP-B-45-14529) and Japanese Unexamined Patent Publication No. 48-30257 (JP-A-48-30257). These processes, however, have a problem in that the reaction therein is a liquid phase reaction and, therefore, a step of separating the catalyst is necessary and, due to use of a mineral acid as the catalyst, the apparatus may be corroded.

With respect to the process of performing the esterification reaction using an acid catalyst in a gaseous phase, for example, U.S. Pat. No. 5,151,547 discloses a production process using a sulfuric acid as a catalyst. According to this process, the problem of the step of separating the catalyst may be solved. However, the problem of corrosion of the apparatus remains because the catalyst used is sulfuric acid which is a mineral acid as in the processes of JP-B-45-14529 and JP-A-48-30257.

On the other hand, Japanese Unexamined Patent Publication No. 57-130954 (JP-A-57-130954) discloses a process of producing a lower aliphatic carboxylic acid ester from a lower aliphatic carboxylic acid and a lower alcohol using a catalyst comprising activated carbon having supported thereon a heteropolyacid or a heteropolyacid salt. According to this patent publication, an extremely strong adsorption is present between the heteropolyacid as an acid catalyst and the activated carbon as a support, therefore, even when the catalyst is used in a liquid phase reaction, the heteropolyacid or a heteropolyacid salt is not dissolved out and the reaction can be continuously performed. The catalyst can also be used as a solid catalyst in a gas phase reaction. Furthermore, it is stated that, in the case of using activated carbon as the support, production of by-products such as an ether, due to dehydration of the lower alcohol, is remarkably inhibited as compared with the case of using other supports and the lower aliphatic carboxylic acid ester can be produced with a high yield. In the Examples thereof, it is verified that the catalyst is advantageous as compared with a catalyst using a silica gel support.

However, the activated carbon has low heat resistance and strength as a support for catalyst, therefore, when a gas phase reaction is continuously performed for a long period of time, the catalyst is finely powdered and causes an increase in the pressure loss within a reactor and, in turn, it becomes difficult to continue the reaction. Furthermore, in the case of performing a reaction in a gas phase using a catalyst comprising activated carbon having supported thereon a heteropolyacid or a heteropolyacid salt, the initial activity of the catalyst is low, therefore, the industrial practice of the reaction is difficult.

### DISCLOSURE OF INVENTION

An object of the present invention is to provide a catalyst, for use in producing a lower aliphatic carboxylic acid ester, which is used in the process of producing a lower aliphatic carboxylic acid ester by esterifying a lower aliphatic carboxylic acid with a lower alcohol in a gas phase and which can ensure excellent initial activity and enable the reaction to be performed stably and continuously for a long period of time. The other objects of the present invention are to provide a process for producing the catalyst and to provide a process for producing a lower aliphatic carboxylic acid ester starting from a lower aliphatic carboxylic acid and a lower alcohol using the catalyst.

As a result of extensive investigations to solve the above-described problems, the present inventors have found that when an esterification reaction of a lower aliphatic carboxylic acid and a lower alcohol is performed in a gas phase using a catalyst for use in producing a lower aliphatic carboxylic acid ester and when the catalyst comprises an inorganic support having supported thereon at least one heteropolyacid and/or heteropolyacid salt, excellent initial activity can be ensured and the reaction can be performed stably and continuously for a long period of time. The present invention has been accomplished based on this finding.

More specifically, the present invention (I) is a catalyst, for use in producing a lower aliphatic carboxylic acid ester, which is used in producing a lower aliphatic carboxylic acid ester from a lower alcohol and a lower aliphatic carboxylic acid in a gas phase, wherein the catalyst comprises an inorganic support having supported thereon at least one heteropolyacid and/or heteropolyacid salt.

The present invention (II) is a process for producing the catalyst, for use in producing a lower aliphatic carboxylic acid ester, of the present invention (I).

The present invention (III) is a process for producing a lower aliphatic carboxylic acid ester, comprising reacting a lower alcohol and a lower aliphatic carboxylic acid in a gas phase in the presence of the catalyst for use in producing a lower aliphatic carboxylic acid ester of the present invention (I).

### BEST MODE FOR CARRYING OUT THE INVENTION

The present invention (I) is a catalyst, for use in producing a lower aliphatic carboxylic acid ester, which is used in producing a lower aliphatic carboxylic acid ester from a lower alcohol and a lower aliphatic carboxylic acid in a gas phase, wherein the catalyst comprises an inorganic support having supported thereon at least one heteropolyacid and/or heteropolyacid salt.

The heteropolyacid for use in the catalyst of the present invention (I) comprises a center element and peripheral elements to which oxygen is connected. The center element is usually silicon or phosphorus but the center element is not limited thereto and may include any one element selected from the elements belonging to Groups 1 to 17 of the Periodic Table. The “Periodic Table” as used herein refers to the Periodic Table according to Kokusai Junsei Oyobi Oyo Kagaku Rengo Muki Kagaku Meimeiho, Kaitei-Ban (Revised Nomenclature in Inorganic Chemistry by International Pure and Applied Science Association) (1989).

Specific examples of the center element include a cupric ion; divalent beryllium, zinc, cobalt and nickel ions; trivalent boron, aluminum, gallium, iron, cerium, arsenic, antimony, phosphorus, bismuth, chromium and rhodium ions; tetravalent silicon, germanium, tin, titanium, zirconium, vanadium, sulfur, tellurium, manganese, nickel, platinum, thorium, hafnium and cerium ions and other rare earth element ions; pentavalent phosphorus, arsenic, vanadium and antimony ions; a hexavalent tellurium ion; and a heptavalent iodide ion, however, the present invention is by no means limited thereto.

Specific examples of the peripheral element include tungsten, molybdenum, vanadium, niobium and tantalum. However, the present invention is not limited thereto.

These heteropolyacids are also known as “polyoxoanions”, “polyoxometallates” or “metal oxide clusters”. The structures of some well-known anions are known, for example, as Keggin, Wells-Dawson or Anderson-Evans-Perloff structures. These are described in detail in Poly-san no Kagaku, Kikan Kagaku Sosetsu (Chemistry of Polyacids, the Introduction of Chemistry Ouarterly), No. 20, edited by Nippon Kagaku Kai (1993). The heteropolyacid usually has a high molecular weight, for example, a molecular weight in the range from 700 to 8,500, and includes not only monomers thereof but also dimeric complexes.

Preferred examples of the heteropolyacid which can be used for the catalyst of the present invention (I) include:

| | |
|---|---|
| tungstosilicic acid | H₄[SiW₁₂O₄₀].xH₂O |
| tungstophosphoric acid | H₃[PW₁₂O₄₀].xH₂O |
| molybdophosphoric acid | H₃[PMo₁₂O₄₀].xH₂O |
| molybdosilicic acid | H₄[SiMo₁₂O₄₀].xH₂O |
| vanadotungstosilicic acid | H₄₊ₙ[SiVₙW₁₂₋ₙO₄₀].xH₂O |
| vanadotungstophosphoric acid | H₃₊ₙ[PVₙW₁₂₋ₙO₄₀].xH₂O |
| vanadomolybdophosphoric acid | H₃₊ₙ[PVₙMo₁₂₋ₙO₄₀].xH₂O |
| vanadomolybdosilicic acid | H₄₊ₙ[SiVₙMo₁₂₋ₙO₄₀].xH₂O |
| molybdotungstosilicic acid | H₄[SiMoₙW₁₂₋ₙO₄₀].xH₂O |
| molybdotungstophosphoric acid | H₃[PMoₙW₁₂₋ₙO₄₀].xH₂O |

wherein n is an integer of 1 to 11 and x is an integer of 1 or more. Of course, the present invention is not limited thereto.

The method for synthesizing these heteropolyacids is not particularly limited and any method may be used. For example, the heteropolyacid can be obtained by heating an acidic aqueous solution (pH: approximately from 1 to 2) containing a salt of molybdic acid or tungstic acid and a simple oxygen acid of hetero atom or a salt thereof. For isolating the heteropolyacid compound from the resulting aqueous heteropolyacid solution, a method of crystallizing and separating the compound as a metal salt may be used. Specific examples thereof are described in Shin Jikken Kagaku Koza 8, Muki Kagobutsuno Gosei (III) (New Experimental Chemistry Course 8, Synthesis (III) of Inorganic Compounds), 3rd ed., edited by Nippon Kagaku Kai, published by Maruzen, page 1413 (Aug. 20, 1984), however, the present invention is not limited thereto. The Keggin structure of the synthesized heteropolyacid can be identified by X-ray diffraction or by UV or IR measurements other than the chemical analysis.

The heteropolyacid salt for use in the catalyst of the present invention (I) is not particularly limited as long as it is a metal salt or onium salt resulting from substituting a part or all of the hydrogen atoms of the heteropolyacid.

Specific examples thereof include metal salts, such as of lithium, sodium, potassium, cesium, magnesium, barium, copper, gold and gallium, and onium salts such as of ammonia, of the above-described heteropolyacids, however, the present invention is not limited thereto.

Among these heteropolyacid salts, preferred are lithium salts, sodium salts, potassium salts, cesium salts, magnesium salts, barium salts, copper salts, gold salts, gallium salts and ammonium salts of the above-described preferred heteropolyacids, and more preferred are lithium salt and cesium salt of tungstosilicic acid and lithium salt and cesium salt of tungstophosphoric acid.

Particularly, when the heteropolyacid is a free acid or comprises several salts, the heteropolyacid has a relatively high solubility in a polar solvent such as water or other oxygen-containing solvents, and the solubility can be controlled by selecting an appropriate counter ion.

Examples of the starting material of the element for forming a heteropolyacid salt in the present invention include lithium nitrate, lithium acetate, lithium sulfate, lithium sulfite, lithium carbonate, lithium phosphate, lithium oxalate, lithium nitrite, lithium chloride, lithium citrate, sodium nitrate, sodium acetate, sodium sulfate, sodium carbonate, monosodium phosphate, disodium phosphate, sodium oxalate, sodium nitrite, sodium chloride, sodium citrate, magnesium nitrate hexahydrate, magnesium acetate tetrahydrate, magnesium sulfate, magnesium carbonate, magnesium phosphate tricosahydrate, magnesium oxalate dihydrate, magnesium chloride, magnesium citrate, barium nitrate, barium acetate, barium sulfate, barium carbonate, barium hydrogenphosphate, barium oxalate monohydrate, barium sulfite, barium chloride, barium citrate, copper nitrate, copper acetate, copper sulfate, copper carbonate, copper diphosphate, copper oxalate, copper chloride, copper citrate, aurous chloride, chloroauric acid, auric oxide, auric hydroxide, auric sulfide, aurous sulfide, gallium dichloride, gallium monochloride, gallium citrate, gallium acetate, gallium nitrate, gallium sulfate, gallium phosphate, ammonium acetate, ammonium carbonate, ammonium nitrate, ammonium dihydrogenphosphate, ammonium hydrogencarbonate, ammonium citrate, ammonium nitrate, diammonium phosphate, monoammonium phosphate and ammonium sulfate. However, the present invention is by no means limited thereto.

Among these, preferred are lithium nitrate, lithium acetate, lithium carbonate, lithium oxalate, lithium citrate, sodium nitrate, sodium acetate, sodium carbonate, sodium oxalate, sodium citrate, copper nitrate, copper acetate, copper carbonate, copper citrate, aurous chloride, chloroauric acid, gallium citrate, gallium acetate and gallium nitrate, and more preferred are lithium nitrate, lithium acetate, lithium carbonate, lithium oxalate, lithium citrate, sodium nitrate, sodium acetate, sodium carbonate, sodium oxalate, sodium citrate, copper nitrate, copper acetate, copper carbonate and copper citrate.

Specific examples of the heteropolyacid salt which can be used in the catalyst, for use in producing a lower aliphatic carboxylic acid ester of the present invention (I), include lithium salt of tungstosilicic acid, sodium salt of tungstosilicic acid, copper salt of tungstosilicic acid, gold salt of tungstosilicic acid, gallium salt of tungstosilicic acid, lithium salt of tungstophosphoric acid, sodium salt of tungstophosphoric acid, copper salt of tungstophosphoric acid, gold salt of tungstophosphoric acid, gallium salt of tungstophosphoric acid, lithium salt of molybdophosphoric acid, sodium salt of molybdophosphoric acid, copper salt of molybdophosphoric acid, gold salt of molybdophosphoric acid, gallium salt of molybdophosphoric acid, lithium salt of molybdosilicic acid, sodium salt of molybdosilicic acid, copper salt of molybdosilicic acid, gold salt of molybdosilicic acid, gallium salt of molybdosilicic acid, lithium salt of vanadotungstosilicic acid, sodium salt of vanadotungstosilicic acid, copper salt of vanadotungstosilicic acid, gold salt of vanadotungsto-silicic acid, gallium salt of vanadotungstosilicic acid, lithium salt of vanadotungstophosphoric acid, sodium salt of vanadotungstophosphoric acid, copper salt of vanadotungstophosphoric acid, gold salt of vanadotungstophosphoric acid, gallium salt of vanadotungstophosphoric acid, lithium salt of vanadomolybdophosphoric acid, sodium salt of vanadomolybdophosphoric acid, copper salt of vanadomolybdophosphoric acid, gold salt of vanadomolybdo-phosphoric acid, gallium salt of vanadomolybdophosphoric acid, lithium salt of vanadomolybdosilicic acid, sodium salt of vanadomolybdosilicic acid, copper salt of vanadomolybdosilicic acid, gold salt of vanadomolybdosilicic acid, gallium salt of vanadomolybdosilicic acid, lithium salt of molybdotungstosilicic acid, sodium salt of molybdotungstosilicic acid, copper salt of molybdotungsto-silicic acid, gold salt of molybdotungstosilicic acid, gallium salt of molybdotungstosilicic acid, lithium salt of molybdotungstophosphoric acid, sodium salt of molybdotungstophosphoric acid, copper salt of molybdotungstophosphoric acid, gold salt of molybdotungstophosphoric acid and gallium salt of molybdotungstophosphoric acid.

Among these, preferred are lithium salt of tungstosilicic acid, sodium salt of tungstosilicic acid, copper salt of tungstosilicic acid, gold salt of tungstosilicic acid, gallium salt of tungstosilicic acid, lithium salt of tungstophosphoric acid, sodium salt of tungstophosphoric acid, copper salt of tungstophosphoric acid, gold salt of tungstophosphoric acid, gallium salt of tungstophosphoric acid, lithium salt of molybdophosphoric acid, sodium salt of molybdophosphoric acid, copper salt of molybdophosphoric acid, gold salt of molybdophosphoric acid, gallium salt of molybdophosphoric acid, lithium salt of molybdosilicic acid, sodium salt of molybdosilicic acid, copper salt of molybdosilicic acid, gold salt of molybdosilicic acid, gallium salt of molybdosilicic acid, lithium salt of vanadotungstosilicic acid, sodium salt of vanadotungstosilicic acid, copper salt of vanadotungsto-silicic acid, gold salt of vanadotungstosilicic acid, gallium salt of vanadotungstosilicic acid, lithium salt of vanadotungstophosphoric acid, sodium salt of vanadotungsto-phosphoric acid, copper salt of vanadotungstophosphoric acid, gold salt of vanadotungstophosphoric acid and gallium salt of vanadotungstophosphoric acid.

More preferred are lithium salt of tungstosilicic acid, sodium salt of tungstosilicic acid, copper salt of tungstosilicic acid, gold salt of tungstosilicic acid, gallium salt of tungstosilicic acid, lithium salt of tungstophosphoric acid, sodium salt of tungstophosphoric acid, copper salt of tungstophosphoric acid, gold salt of tungstophosphoric acid, gallium salt of tungstophosphoric acid, lithium salt of vanadotungstosilicic acid, sodium salt of vanadotungstosilicic acid, copper salt of vanadotungstosilicic acid, gold salt of vanadotungstosilicic acid, gallium salt of vanadotungstosilicic acid, lithium salt of vanadotungstophosphoric acid, sodium salt of vanadotungstophosphoric acid, copper salt of vanadotungstophosphoric acid, gold salt of vanadotungstophosphoric acid and gallium salt of vanadotungstophosphoric acid.

In the catalyst of the present invention (I), two or more selected from the group consisting of these heteropolyacids and/or heteropolyacid salts may also be used.

The catalyst, for use in producing a lower aliphatic carboxylic acid ester of the present invention (I), is a so-called supported catalyst in which a heteropolyacid and/or heteropolyacid salt as the catalyst component is supported on an inorganic support. Examples of the inorganic support which can be used include inorganic supports such as silica, alumina, silica alumina and zeolite. These inorganic supports are superior to other organic supports in heat resistance and strength under the conditions of the production process of a lower aliphatic carboxylic acid ester, which is described later, and can stably maintain the catalytic activity for a long period of time in industrial practice.

Among these inorganic supports, silica is preferred because when silica is used as the inorganic support, the catalyst, for use in producing a lower aliphatic carboxylic acid ester, exhibits particularly high activity in the esterification reaction. The term “silica” as used herein means an inorganic support mainly comprising SiO₂. Among these, preferred is silica gel having an SiO₂ content of 90% by mass or more, more preferably 95% by mass or more, based on the entire mass of the inorganic support.

The SiO₂ content in silica gel may be measured by any method and examples thereof include the following method using a hydrofluoric acid, more specifically, a measurement method comprising the following steps 1) to 4):

1) about 1 g of a sample after drying at 170° C. in air or at 150° C. in vacuum for 2 hours is measured to an accuracy of 0.1 mg;

2) the sample is wetted with water and twice subjected to an operation of adding a few drops of sulfuric acid and about 20 cm³ of hydrofluoric acid, and heating and evaporating the sample on a sand bath;

3) the sample is heated at 1,000° C. for 5 minutes and allowed to cool in a desiccator and then the weight of residue is measured; and

4) from the difference in weight, between before and after these steps, the SiO₂ content is calculated.

In the case where a heteropolyacid and/or heteropolyacid salt as the catalyst component is already supported, after removing the supported components by water washing, the SiO₂ content in silica gel can be measured by the above-described method. This is described in detail in JIS K 1150. Needless to say, the measurement method is not limited thereto and commonly used measurement methods may also be used.

The silica gel used as the inorganic support of the catalyst, for use in producing the lower aliphatic carboxylic acid ester of the present invention (I), may contain any component as long as it does not inhibit the esterification reaction in the process for producing a lower aliphatic carboxylic acid ester, which process is characterized by reacting a lower alcohol and a lower aliphatic carboxylic acid in a gas phase. In general, silica gel used as a support for catalysts contains various elements and in the case of using silica gel as the support of the catalyst of the present invention (I), any component may be contained therein as long as it does not inhibit the reaction.

Specific examples of the elements which are generally contained in silica gel include potassium, sodium, calcium, chromium, iron, magnesium, cobalt, nickel, copper, zirconium, titanium, aluminum, strontium, niobium and rubidium. The inorganic support used for the catalyst of the present invention (I) may contain any of these components.

However, in the case of obtaining a catalyst comprising only a heteropolyacid as the catalyst component (namely, completely free of a heteropolyacid salt), it is necessary to use a support not containing elements able to form a heteropolyacid salt. The reason is that the heteropolyacid may form a salt with the elements contained in the support and a catalyst comprising only a heteropolyacid is substantially impossible to obtain. In other words, the term “a heteropolyacid salt” as used in the present invention also includes salts formed with elements in the support.

The shape of the inorganic support for use in the catalyst of the present invention (I) is not particularly limited and the inorganic support may be a powder, or in a spherical, pellet-like or any other arbitrary shape according to the reaction form used. The suitable average diameter of the inorganic support varies depending on the reaction form. However, in the case of a fixed bed reaction, the average diameter is suitably from 2 to 10 mm, preferably from 3 to 7 mm and, in the case of a fluidized bed reaction, the average diameter is suitably from a powder to 5 mm, preferably from a powder to 2 mm.

The inorganic support suitably has a specific surface area such that a catalyst obtained after loading a heteropolyacid and/or a heteropolyacid salt on the inorganic support has a specific surface area, by the BET method, of 65 to 350 m²/g, preferably from 80 to 300 m²/g, more preferably from 100 to 250 m²/g.

With respect to the amount of the heteropolyacid and/or the heteropolyacid salt for use in the catalyst of the present invention (I), the sum total of heteropolyacid and/or heteropolyacid salt supported is suitably from 50 to 1,000 g, preferably from 100 to 800 g, more preferably from 150 to 600 g, based on 1 liter of the inorganic support before it is loaded with heteropolyacid and/or heteropolyacid salt.

If the supported amount of the heteropolyacid and/or heteropolyacid salt is less than 50 g based on 1 liter of the inorganic support before it is loaded with the catalyst component, the content of the catalyst component is small and, therefore, the activity for the objective esterification may seriously decrease and selectivity for ethers as by-products may increase, whereas if the supported amount of the heteropolyacid and/or heteropolyacid salt exceeds 1,000 g based on 1 liter of the inorganic support before the loading of the catalyst component, the catalyst may be reduced in the effective surface area and, due to coking, covering of active sites or blocking of catalyst pores readily occurs to seriously shorten the catalyst life.

The amount of the heteropolyacid and/or heteropolyacid salt in the catalyst of the present invention (I) can be determined by analyzing the amounts of constituent elements such as tungsten and molybdenum contained in the heteropolyacid and/or heteropolyacid salt using inductively coupled plasma emission spectrometry (hereinafter referred to as “ICP”), a fluorescent X-ray spectrometry or an atomic absorption spectrometry. Specific examples of the measuring method include a method of dissolving the catalyst using an acid such as hydrochloric acid, nitric acid, sulfuric acid or hydrofluoric acid or using a mixed acid of two or more thereof, measuring ICP spectral line intensities of molybdenum (wavelength: 386.40 nm) and tungsten (wavelength: 276.43 nm), and performing the quantitative analysis using a calibration curve method which uses a standard sample. This is described in detail in JIS G 1258 and Bunseki Kagaku Binran (Analysis Chemistry Handbook), 3rd ed., compiled by Nippon Bunseki Kagaku Kai, issued by Maruzen.

The process for producing a catalyst for use in producing a lower aliphatic carboxylic acid ester, of the present invention (II), is described below.

The catalyst of the present invention is, as described above, roughly classified into the following two groups;

A) a catalyst containing at least one salt of heteropolyacid (including both a metal salt or an onium salt resulting from substituting a part of hydrogen atoms of heteropolyacid and a salt resulting from substituting all of the hydrogen atoms) (hereinafter simply referred to as a “salt catalyst”), and

B) a catalyst completely free of a heteropolyacid salt (hereinafter simply referred to as a “free catalyst”). The production processes of these catalysts are different from each other.

The production process of the salt catalyst includes the following three kinds of processes (1) to (3), namely,

(1) a process for producing a catalyst, for use in producing a lower aliphatic carboxylic acid ester, comprising the following first and second steps:

First Step

a step of loading a heteropolyacid on an inorganic support to obtain a heteropolyacid supported catalyst; and

Second Step

a step of loading an element for forming a salt on the heteropolyacid supported catalyst obtained in the first step to produce a catalyst for use in producing a lower aliphatic carboxylic acid ester;

(2) a process for producing a catalyst, for use in producing a lower aliphatic carboxylic acid ester, comprising a step of loading a heteropolyacid together with a starting material for the element for forming a salt or loading a previously prepared heteropolyacid salt on an inorganic support; and

(3) a process for producing a catalyst, for use in producing a lower aliphatic carboxylic acid ester, comprising the following first and second steps:

First Step

a step of loading a starting material for the element of forming a salt of heteropolyacid on an inorganic support to obtain a salt-forming component supporting support; and

Second Step

a step of loading a heteropolyacid on the salt-forming component supporting support obtained in the first step to obtain a catalyst for use in producing a lower aliphatic carboxylic acid ester.

In any of these processes (1) to (3), the heteropolyacid and the starting material for the element of forming a heteropolyacid salt each can be loaded on an inorganic support after dissolving or suspending it in an appropriate solvent. The solvent may be any as long as it can uniformly dissolve or suspend the desired heteropolyacid, a heteropolyacid salt and the starting material for the element for forming a salt, and examples of the solvent which can be used include water, an organic solvent and a mixture thereof. Among these, preferred are water, alcohols and carboxylic acids.

The method used for the dissolution or suspension may also be any as long as it can uniformly dissolve or suspend the desired heteropolyacid, a heteropolyacid salt and the starting material for the element for forming a salt. In the case of a free acid, a free acid which can dissolve may be dissolved as it is in a solvent and even in the case of a free acid which cannot completely dissolve, if the free acid can be uniformly suspended by forming it into fine powder, the free acid may be suspended as such.

In the process (1), a solution or suspension obtained by dissolving or suspending a heteropolyacid in a solvent is absorbed to an inorganic support to thereby load the heteropolyacid on the inorganic support and then, a solution or suspension of a starting material for the element for forming a desired salt is absorbed to the inorganic support to thereby load the element. At this time, a neutralization reaction proceeds on the inorganic support, as a result, a heteropolyacid salt supported catalyst can be prepared.

In the process (2), a heteropolyacid and a starting material for the element of forming a salt are dissolved or suspended together or separately and then mixed to prepare a uniform solution or suspension, and the solution or suspension is absorbed to an inorganic support, thereby loading the heteropolyacid and the element. If the compound is in the state of a heteropolyacid salt, a uniform solution or suspension may be obtained in the same manner as in the case of a free acid.

In the process (3), a solution or suspension of a starting material for the element of forming a salt is previously prepared, the solution or suspension is absorbed to an inorganic support to thereby load the element, and then a desired heteropolyacid is loaded. This method includes a method of using an element which is previously contained in the inorganic support and which can form a heteropolyacid salt.

More specifically, a part or all of the elements previously contained in an inorganic support sometimes act to form a salt of heteropolyacid when the heteropolyacid is loaded, and as a result, a heteropolyacid salt is formed. Examples of such an element include potassium, sodium, calcium, iron, magnesium, titanium and aluminum, however, the present invention is not limited thereto.

The kind of the element previously contained in an inorganic support and the amount thereof can be measured by chemical analysis such as ICP, a fluorescent X-ray spectrometry and an atomic absorption spectrometry. The kind and the amount of the element vary depending on the inorganic support, however, potassium, sodium, calcium, iron, magnesium, titanium and ammonium are sometimes contained in a relatively large amount and the content thereof is approximately from 0.001 to 5.0% by mass. Therefore, depending on the combination of an inorganic support and a heteropolyacid, the element previously contained in the inorganic support may be in an amount large enough to form a salt, though this may vary depending on the kind and the amount of the heteropolyacid supported.

The method for loading a solution or suspension of heteropolyacid or a heteropolyacid salt on an inorganic support is not particularly limited and a known method may be used. More specifically, for example, the catalyst may be prepared by dissolving a heteropolyacid in distilled water corresponding to the liquid absorption amount of an inorganic support used and impregnating the solution into the inorganic support. Also, the catalyst may be prepared using an excess aqueous solution by impregnating it into an inorganic support while appropriately moving the support in the heteropolyacid solution and then removing the excess acid by filtration. The volume of the solution or suspension used at this time varies depending on the inorganic support or loading method used.

The thus-obtained wet catalyst is suitably dried by placing it in a heating oven for a few hours. The drying method is not particularly limited and any method such as standing or a belt conveyor may be used. After the drying, the catalyst is preferably cooled to the ambient temperature in a desiccator so as not to absorb moisture.

On the other hand, the free catalyst may be obtained by the following production process, that is, a process for producing a catalyst, for use in producing a lower aliphatic carboxylic acid ester, comprising a step of loading a heteropolyacid on an inorganic support.

The free catalyst is a catalyst obtained by loading a heteropolyacid on an inorganic support and this can be produced by performing the first step in the process (1) for producing a catalyst. This process is specifically described above. However, since the free catalyst is a catalyst which does not contain a heteropolyacid salt at all as described above, the inorganic support used in this process for producing a catalyst must not contain an element able to form a salt of heteropolyacid.

The amount of the heteropolyacid supported in the heteropolyacid supported catalyst obtained by the production process of the present invention can be simply calculated by subtracting the weight of the inorganic support used from the weight after drying of the catalyst prepared. To be more exactly, the supported amount can be determined by chemical analysis such as ICP, fluorescent X-ray spectrometry or atomic absorption spectrometry.

The present invention (III) is described below.

The present invention (III) is a process, for producing a lower aliphatic carboxylic acid ester, comprising reacting a lower alcohol with a lower aliphatic carboxylic acid in a gas phase in the presence of the catalyst for use in producing a lower aliphatic carboxylic acid ester of the present invention (I).

Examples of the lower alcohol which can be used in the process for producing a lower aliphatic carboxylic acid ester of the present invention (III) include methanol, ethanol, n-propanol, isopropanol, n-butanol, sec-butanol, isobutanol, tert-butanol, allyl alcohol and crotyl alcohol.

The lower aliphatic carboxylic acid is suitably a carboxylic acid having from 1 to 4 carbon atoms. Specific examples thereof include formic acid, acetic acid, propionic acid, butyric acid, acrylic acid and (meth)acrylic acid.

In the esterification reaction of these starting materials using the catalyst of the present invention (I), an esterification reaction of a lower alcohol and a lower aliphatic carboxylic acid is the main reaction. However, depending on the reaction conditions, lower olefins or ethers are produced as a dehydration product of the lower alcohol due to the side reaction shown below. When the lower alcohol is methanol, a corresponding olefin is not present, therefore, the side reaction I does not occur.

Side Reaction I

R—OH→Olefin+H₂O

Side Reaction II

2R—OH→R—O—R+H₂O

From the equilibrium aspect, the production of and selectivity for these by-products may be suppressed by allowing water to be present in the reaction system. However, as concerns this reaction, the esterification reaction which is the main reaction is also a dehydration reaction, therefore, when water is allowed to be present, the activity of the main reaction also decreases, in general.

Nevertheless, in the reaction using the catalyst of the present invention (I), by allowing water within a certain range to be present in the reaction system, the production of by-products can be suppressed while maintaining the activity of the main reaction and also, the catalytic activity can be maintained.

Specifically, the amount of water allowed to be present is, in terms of water concentration in the starting materials, preferably from 1 to 10 mol %, more preferably from 2 to 8 mol %. If the amount of water added is less than 1 mol %, not only the effect of reducing the production of by-products decreases but also a polymerization product of the lower olefin is produced as a by-product and disadvantageously causes lowering of the catalytic activity. Furthermore, since the reaction of producing the objective lower aliphatic carboxylic acid ester is an equilibrium reaction, water present in excess of 10 mol % adversely affects the production of the lower aliphatic carboxylic acid ester and decreases the activity.

The water added in this reaction is not limited to water newly fed but water produced by the esterification may be entirely or partially recovered and used by recycling.

The by-products, as dehydration products of the lower alcohol shown in the side reactions I and II, can produce, when fed back to the reaction system, a lower aliphatic carboxylic acid ester under the reaction conditions in the process for producing a lower aliphatic carboxylic acid ester of the present invention. The reaction routes therefor are considered to include the following reaction I and reaction II:

Reaction I

Olefin+R′COOH→R′COOR

(wherein R represents a group derived from olefin)

Reaction II

R—O—R+2R′COOH→2R′COOR+H₂O

Accordingly, when the lower olefin or ether as a by-product produced by this reaction is separated from the lower aliphatic carboxylic acid ester produced and then recycled to the reaction system, the selectivity of the reaction can, prima facie, be approximately 100%. Recycling is generally preferred from the aspect of production factors. The recycling method is not particularly limited and any method may be used as long as the by-products can be fed to the reactor. Specific examples thereof include a method of recycling the by-products by mixing them into a lower alcohol which is newly fed to the reactor. Of course, the present invention is not limited thereto.

In the process for producing a lower aliphatic carboxylic acid ester of the present invention, it is important to use the lower alcohol and the lower aliphatic carboxylic acid as the starting materials such that the lower aliphatic carboxylic acid is in an equimolar amount or excess molar amount. The reason is that, if the lower alcohol is used in excess, production of the lower olefin or ether due to the above-described side reactions, particularly ether produced from two molecules of lower alcohol, increases and this adversely affects the selectivity and the catalytic activity.

In the esterification reaction itself, the lower alcohol and the lower aliphatic carboxylic acid undertake an equimolar reaction, however, on taking into account the separation by distillation of the objective lower aliphatic carboxylic acid ester from the lower alcohol and the lower aliphatic carboxylic acid as the starting materials, it is generally not easy to separate the lower aliphatic carboxylic acid ester from the lower alcohol because these, in many cases, have similar boiling points.

For example, in the process of producing ethyl acetate from ethanol and acetic acid, ethanol and ethyl acetate have similar boiling points and moreover, these are azeotropic, therefore, ethanol and ethyl acetate cannot be separated by simple distillation which is usually employed as a separation method in industry. Accordingly, in the separation by distillation, the separation must be performed, for example, by a method of adding water at the distillation, dividing the azeotropic fraction into an oil layer and an aqueous layer, separating ethanol and ethyl acetate from the oil layer and, while recirculating a certain amount of the aqueous layer into the distillation tower, extracting a part thereof to recover ethanol.

In any case, as the residual ratio of the starting material lower alcohol is higher, the separation from the lower aliphatic carboxylic acid ester produced becomes difficult and, therefore, the conversion of the lower alcohol is preferably higher. In an actual practice in industry, the conversion of the lower alcohol is preferably at least 70% by mass or more, more preferably 80% by mass or more.

The term “conversion” as used herein means the ratio of the lower alcohol consumed in the esterification reaction. More specifically, the conversion includes not only the change into the lower aliphatic carboxylic acid ester, as the objective product, but also includes changes into lower olefin or ether produced by the side reaction, particularly ether produced from two molecules of lower alcohol, and changes into other by-products or decomposition products.

The ester-producing reaction is generally an equilibrium reaction and the upper limit of the conversion is mostly governed by the equilibrium composition. Therefore, the catalyst and the reaction conditions must be actually selected to give an equilibrium composition having a high conversion of lower alcohol.

However, if too much of the lower aliphatic carboxylic acid is used, the reaction product contains a large amount of unreacted lower aliphatic carboxylic acid and this causes a problem that the energy necessary, for separating and recovering the unreacted lower aliphatic carboxylic acid and recycling it into the reaction system, increases.

Accordingly, the lower alcohol and the lower aliphatic carboxylic acid as the starting materials are preferably fed to the reaction system in a ratio of 1:10 to 1:1 by mol, more preferably from 1:4 to 1:1 by mol. The lower alcohol and the lower aliphatic carboxylic acid as starting materials used here are of course the starting materials newly fed but the present invention is not limited thereto, and the unreacted starting materials separated and recovered in the purification step from the lower aliphatic carboxylic acid ester produced by the reaction may also be entirely or partly used by recycling.

The method for separating unreacted starting materials, namely, lower alcohol and lower aliphatic carboxylic acid, and also separating added water and a lower olefin or ether as by-products from the lower aliphatic carboxylic acid ester produced is not particularly limited and may be freely selected from unit operations such as distillation, extraction, absorption, adsorption, membrane separation and phase separation, by taking account of energy necessary for the separation, ease of the separation or the simplicity and convenience of the equipment. These operations may be used in combination of two or more thereof.

In the process for producing a lower aliphatic carboxylic acid ester of the present invention, the reaction temperature is not particularly limited as long as the medium fed to the reactor is in the gas form, namely, the temperature is higher than the dew point of the mixed gas. The reaction temperature is generally selected in the range from 100 to 250° C., preferably from 120 to 220° C. In view of the reaction rate, if the temperature is low, the reaction rate decreases and can hardly be approximated to the equilibrium conversion. On the other hand, as the temperature becomes higher, an increase in the reaction rate of the side reaction greatly surpasses the increase in the reaction rate of the main reaction and this causes the reduction of selectivity and adversely affects the reaction results.

With respect to the reaction pressure, since the medium fed to the reactor must be in the gaseous form, similarly to the temperature, it is important to select a preferred pressure from a curve showing the relationship between the temperature suitable for reaction, the temperatures of starting materials, namely, lower alcohol and lower aliphatic carboxylic acid, and the temperature of water with the vapor pressure. In view of the reaction rate, if the pressure falls, the reaction rate decreases and, furthermore, with the progress of dehydration reaction of the lower alcohol shown in Side Reaction I, the selectivity decreases. On the other hand, if the pressure increases, the reaction rate increases and can be easily approximated to the equilibrium conversion, however, the dew point of the mixture of the starting materials lower alcohol and lower aliphatic carboxylic acid with water elevates and, therefore, a high reaction temperature is required but this causes a reduction in the selectivity, as described above. Although it may vary depending on the kinds of the starting materials, generally, the reaction pressure is preferably from 0.0 to 3.0 MPaG (gauge pressure), more preferably from 0.0 to 2.0 MPaG (gauge pressure).

The gas hourly space velocity (hereinafter simply referred to as “GHSV”) of the starting materials fed to the reactor is not particularly limited, however, if the GHSV is small, the production of aliphatic carboxylic acid ester produced within a unit time per unit volume of catalyst, the so-called space time yield (hereinafter simply referred to as “STY”), decreases and, as a result, the productivity lowers. If the GHSV is increased, the conversion in single pass decreases and can hardly be approximated to the equilibrium conversion. The STY increases nearly in proportion to GHSV at the beginning, however, if the GHSV is excessively increased, the STY does not increase any more and the effect duly expected from the equipment or the operation cost necessary for increasing the GHSV cannot be obtained. In view of this, the GHSV, in practice, has an optimal range, more specifically, the starting materials are preferably fed to the reaction system at 100 to 7,000/hr, more preferably from 300 to 3,000/hr.

The reaction form is not particularly limited as long as the reaction is performed in a gas phase and any form may be freely selected from the reaction forms such as fixed bed, moving bed and fluidized bed, while taking into account the elimination of the reaction heat, control of the reactor, and simplicity and convenience of the equipment. In the case where the reaction heat is small and scarcely has an effect on the control of reaction, an adiabatic reactor, for example, a fixed bed tank-type reactor, is used in many cases because of simplicity and convenience of the equipment. As the reaction heat becomes larger, a multi-tubular reactor type of fixed bed reactor, moving bed reactor or a fluidized bed reactor is generally used so as to keep the catalyst layer at a uniform temperature. These are, however, only representative examples and the reaction form is not limited thereto.

The present invention is described in greater detail below by referring to the Examples and Comparative Examples, however, these Examples are described to show an outline of the present invention and the present invention should not be construed as being limited thereto.

<Analysis of Reaction Gas>

In Examples and Comparative Examples, the starting material composition fed to the reactor was used as the inlet gas concentration. The gas at the outlet of the reactor was entirely cooled and the concentrated reaction solution collected was recovered in the whole amount and analyzed by gas chromatography. With respect to the effluent gas remaining uncondensed, the whole amount of the uncondensed gas flowing out within the sampling time was measured and a part of the gas was sampled and analyzed on the composition by gas chromatography. The analysis conditions are shown below.

Conditions for Analysis of Uncondensed Gas

An absolute calibration curve method was used for the analysis. The analysis was performed, under the following conditions, by sampling 50 ml of the effluent gas and passing the whole amount thereof into a 1 ml-volume gas sampler attached to the gas chromatograph.

1. Ether, Lower Aliphatic Carboxylic Acid Ester, Lower Alcohol, Lower Aliphatic Carboxylic Acid, Trace By-Products

Gas Chromatography:

gas chromatograph (GC-14B, manufactured by Shimadzu Seisakusho Co.) with a gas sampler (MGS-4, measuring tube: 1 ml) for Shimadzu gas chromatograph

Column:

packed column SPAN80 15% Shinchrom A, 60 to 80 mesh (length: 5 m)

Carrier Gas: Nitrogen (Flow Rate: 25 ml/min)

Temperature Conditions:

The detector and the vaporization chamber were at a temperature of 120° C. and the column temperature was 65° C. and constant.

Detector:

FID (H₂ pressure: 60 kPaG, air pressure: 100 kPaG)

2. Butene

Gas Chromatography:

gas chromatograph (GC-14B, manufactured by Shimadzu Seisakusho Co.) with a gas sampler (MGS-4, measuring tube: 1 ml) for Shimadzu gas chromatograph

Column: Packed Column Unicarbon A-400, Length: 2 m

Carrier Gas: Helium (Flow Rate: 23 ml/min)

Temperature Conditions:

The detector and the vaporization chamber were constantly at a temperature of 130° C. and the column temperature was elevated from 40° C. to 90° C. at a temperature increasing rate of 40° C./min.

Detector:

FID (H₂ pressure: 70 kPaG, air pressure: 100 kPaG) Butene as a representative of ethylene oligomers was measured during production.

3. Ethylene

Gas Chromatography:

gas chromatograph (GC-14B, manufactured by Shimadzu Seisakusho Co.) with a gas sampler (MGS-4, measuring tube: 1 ml) for Shimadzu gas chromatograph

Column: Packed Column Unibeads IS, Length: 3 m

Carrier Gas: Helium (Flow Rate: 20 ml/min)

Temperature Conditions:

The detector and the vaporization chamber were at a temperature of 120° C. and the column temperature was 65° C. and constant.

Detector:

TCD (He pressure: 70 kPaG, current: 90 mA, temperature: 120° C.)

Analysis of Solution Collected

The analysis was performed using the internal standard method, where the analysis solution was prepared by adding 1 ml of 1,4-dioxane as the internal standard to 10 ml of the reaction solution and 0.2 μl of the analysis solution was injected.

Gas Chromatography:

gas chromatograph (GC-14B, manufactured by Shimadzu Seisakusho Co.)

Column:

capillary column TC-WAX (length: 30 m, internal diameter: 0.25 mm, film thickness: 25 μm)

Carrier Gas:

nitrogen (split ratio: 20, column flow rate: 1 ml/min)

Temperature Conditions:

The detector and the vaporization chamber were constantly at a temperature of 200° C. and the column temperature was kept at 40° C. for 7 minutes from the initiation of the analysis, thereafter elevated up to 230° C. at a temperature increasing rate of 10° C./min, and kept at 230° C. for 10 minutes.

Detector:

FID (H₂ pressure: 70 kPaG, air pressure: 100 kPaG)

<Support>

Support 1:

synthetic silica gel (CARiACT Q-10, produced by Fuji Silicia Kagaku K.K.) (specific surface area: 219.8 m²/g, pore volume: 0.660 cm³/g) (results of fluorescent X-ray spectrometry analysis, purity: 99.97%, Fe₂O₃: 0.03%)

Support 2:

natural silica gel (KA-160, produced by Sud Chemie A G) (specific surface area: 130 m²/g, pore volume: 0.53 cm³/g) (results of fluorescent X-ray analysis, purity: 98.10%, Fe₂O₃: 0.68%, TiO₂: 0.67%, K₂O: 0.34%, CaO: 0.16%, ZrO₂: 0.05%)

Support 3:

activated carbon (particulate SHIRASAGI Cx, 4 to 6 mesh, produced by Takeda Yakuhin Kogyo K.K.) (specific surface area: 544.7 m²/g, pore volume: 0.298 cm³/g)

<Preparation of Catalyst>

The support used for each catalyst was dried for 4 hours in a hot air dryer adjusted to 110° C. In the case of loading a heteropolyacid, a predetermined amount of heteropolyacid was weighed and in the case of loading a partially neutralized salt of heteropolyacid, a predetermined amount of heteropolyacid and a predetermined amount of a metal nitrate for the neutralization were weighed. Thereto, 15 ml of pure water was added and the mixture was uniformly dissolved to obtain an impregnating solution. In this impregnating solution, 100 ml of the support was placed and thoroughly stirred. The support impregnated with the solution was air dried for 1 hour and thereafter dried for 5 hours by a dryer adjusted to 150° C. to obtain a catalyst. The thus-prepared catalysts are shown together in Table 1.

TABLE 1

| | | | Catalyst Components | | |
|---|---|---|---|---|---|
| | | | | | Amount of |
| | | | Amount of | | Neutralization |
| | Support | Hetero- | Hetero- | Neutralization | Component*1 |
| | Kind | polyacid | polyacid [g] | Component | [g] |
| Catalyst 1 | Support 1 | H₄SiW₁₂O₄₀ | 35.0 | — | — |
| Catalyst 2 | Support 1 | H₃PW₁₂O₄₀ | 55.0 | — | — |
| Catalyst 3 | Support 1 | H₄SiW₁₂O₄₀ | 4.0 | — | — |
| Catalyst 4 | Support 2 | H₄SiW₁₂O₄₀ | 35.0 | LiNO₃ | 0.083 |
| Catalyst 5 | Support 2 | H₄SiW₁₂O₄₀ | 35.0 | NaNO₃ | 0.103 |
| Catalyst 6 | Support 2 | H₃PW₁₂O₄₀ | 55.0 | LiNO₃ | 0.132 |
| Catalyst 7 | Support 2 | H₃PW₁₂O₄₀ | 55.0 | KNO3 | 0.193 |
| Catalyst 8 | Support 2 | H₃PW₁₂O₄₀ | 55.0 | NaNO₃ | 0.162 |
| Catalyst 9 | Support 3 | H₄SiW₁₂O₄₀ | 35.0 | — | — |
| Catalyst 10 | Support 3 | H₄SiW₁₂O₄₀ | 35.0 | LiNO₃ | 0.083 |

*1 The amount of neutralization component is a value as a nitrate

### EXAMPLES 1 to 18 and COMPARATIVE EXAMPLES 1 and 2

Into a reaction tube, 40 ml of each catalyst shown in Table 1 was filled and the reaction was performed under the reaction conditions shown in Table 2 (temperature, pressure, GHSV, molar ratio of starting materials). The reaction mixture obtained at the outlet of reaction tube was analyzed on the components by the above-described method and the reaction result was calculated. The results are shown in Table 3.

In Example 1, Example 4, Comparative Example 1 and Comparative Example 2, the reaction was further continuously performed for 500 hours under the conditions shown in Table 2. After the completion of reaction, the catalyst was taken out and fine powder was removed through a 60-mesh sieve. From the mass ratio between the amount of the catalyst filled and the amount of the catalyst after fine powder was removed, the retention in percentage of the catalyst shape was calculated and used as an index for the durability of catalyst. The results are shown in Table 3.

It is apparent that in Comparative Example 9 and Comparative Example 10 for Catalyst 9 and Catalyst 10 using activated carbon as the support, the reaction activity and the durability of catalyst were inferior.

TABLE 2

| | | Reaction | Reaction | | Molar Ratio of Starting Materials | | | | | |
|---|---|---|---|---|---|---|---|---|---|---|
| | | Temperature | Pressure | GHSV | | Acetic | | | | |
| | Catalyst | [° C.] | [MPaG] | [1/h] | Ethanol | Acid | Water | Nitrogen | Ethylene | DEE |
| Example 1 | Catalyst 1 | 165 | 0.8 | 1500 | 4.0 | 8.0 | 4.5 | 83.5 | — | — |
| Example 2 | Catalyst 2 | 165 | 0.8 | 1500 | 4.0 | 8.0 | 4.5 | 83.5 | — | — |
| Example 3 | Catalyst 3 | 165 | 0.8 | 1500 | 4.0 | 8.0 | 4.5 | 83.5 | — | — |
| Example 4 | Catalyst 4 | 165 | 0.8 | 1500 | 4.0 | 8.0 | 4.5 | 83.5 | — | — |
| Example 5 | Catalyst 5 | 165 | 0.8 | 1500 | 4.0 | 8.0 | 4.5 | 83.5 | — | — |
| Example 6 | Catalyst 6 | 165 | 0.8 | 1500 | 4.0 | 8.0 | 4.5 | 83.5 | — | — |
| Example 7 | Catalyst 7 | 165 | 0.8 | 1500 | 4.0 | 8.0 | 4.5 | 83.5 | — | — |
| Example 8 | Catalyst 8 | 165 | 0.8 | 1500 | 4.0 | 8.0 | 4.5 | 83.5 | — | — |
| Example 9 | Catalyst 4 | 165 | 0.8 | 1500 | 4.0 | 10.0 | 4.5 | 81.5 | — | — |
| Example 10 | Catalyst 4 | 165 | 0.8 | 1500 | 5.0 | 5.0 | 4.5 | 85.5 | — | — |
| Example 11 | Catalyst 4 | 165 | 0.8 | 1500 | 4.0 | 8.0 | 4.5 | 83.5 | — | — |
| Example 12 | Catalyst 4 | 165 | 0.8 | 1500 | 4.0 | 8.0 | 4.5 | 81.5 | 1.0 | 1.0 |
| Example 13 | Catalyst 4 | 165 | 0.8 | 1500 | 4.0 | 8.0 | 4.5 | 81.5 | 1.0 | 1.0 |
| Example 14 | Catalyst 4 | 270 | 0.8 | 1500 | 4.0 | 8.0 | 4.5 | 83.5 | — | — |
| Example 15 | Catalyst 4 | 165 | 0.8 | 10000 | 4.0 | 8.0 | 4.5 | 83.5 | — | — |
| Example 16 | Catalyst 4 | 165 | 0.8 | 50 | 4.0 | 8.0 | 4.5 | 83.5 | — | — |
| Example 17 | Catalyst 4 | 165 | 0.8 | 1500 | 4.0 | 8.0 | 0.0 | 88.0 | — | — |
| Example 18 | Catalyst 4 | 165 | 0.8 | 1500 | 4.0 | 8.0 | 0.0 | 81.5 | 1.0 | 1.0 |
| Comparative | Catalyst 9 | 165 | 0.8 | 1500 | 4.0 | 8.0 | 4.5 | 83.5 | — | — |
| Example 1 | | | | | | | | | | |
| Comparative | Catalyst 10 | 165 | 0.8 | 1500 | 4.0 | 8.0 | 4.5 | 83.5 | — | — |
| Example 2 | | | | | | | | | | |

DDE: diethyl ether

TABLE 3

| | | | | | | | | | | | Retention in |
|---|---|---|---|---|---|---|---|---|---|---|---|
| | Conversion [%] | | Reaction Product STY [g/l · h] | | | | Selectivity [%] | | | | Percentage of |
| | | Acetic | Ethyl | | | | Ethyl | | | | Catalyst Shape |
| | Ethanol | Acid | Acetate | DEE | Ethylene | Butene | Acetate | DEE | Ethylene | Butene | [%] |
| Example 1 | 84.2 | 41.0 | 172.0 | 2.0 | 2.9 | 0.0015 | 88.7 | 4.9 | 6.7 | 0.0034 | 95.8 |
| Example 2 | 81.5 | 39.9 | 163.0 | 2.1 | 3.0 | 0.0017 | 85.3 | 6.7 | 8.0 | 0.0048 | — |
| Example 3 | 17.8 | 8.9 | 49.0 | 0.7 | 0.8 | 0.0001 | 94.1 | 2.1 | 3.8 | 0.0003 | — |
| Example 4 | 92.6 | 46.9 | 205.0 | 2.2 | 3.5 | 0.0027 | 92.1 | 2.4 | 5.6 | 0.0050 | 96.6 |
| Example 5 | 88.2 | 42.8 | 176.0 | 5.5 | 7.4 | 0.0105 | 79.8 | 10.0 | 10.2 | 0.0293 | |
| Example 6 | 95.1 | 45.1 | 224.0 | 4.8 | 6.2 | 0.0087 | 87.1 | 5.0 | 7.9 | 0.0092 | — |
| Example 7 | 91.1 | 42.1 | 196.0 | 3.1 | 3.3 | 0.0071 | 90.7 | 4.6 | 4.7 | 0.0110 | — |
| Example 8 | 88.9 | 41.8 | 188.0 | 2.2 | 3.0 | 0.0066 | 92.2 | 3.5 | 4.3 | 0.0081 | — |
| Example 9 | 94.0 | 40.5 | 216.0 | 1.3 | 2.6 | 0.0019 | 95.0 | 1.4 | 3.6 | 0.0020 | — |
| Example 10 | 73.5 | 70.2 | 175.0 | 6.8 | 8.1 | 0.0208 | 84.3 | 6.6 | 9.1 | 0.0310 | — |
| Example 11 | 92.5 | 45.7 | 201.0 | 3.3 | 4.5 | 0.0058 | 90.5 | 3.4 | 6.1 | 0.0072 | — |
| Example 12 | 88.2 | 43.2 | 190.0 | 6.2 | 7.3 | 0.1480 | 85.1 | 6.3 | 8.6 | 0.0621 | — |
| Example 13 | 90.5 | 46.3 | 208.0 | 3.5 | 4.6 | 0.1024 | 98.3 | 0.6 | 1.1 | 0.0041 | — |
| Example 14 | 92.1 | 45.7 | 160.0 | 12.5 | 10.3 | 0.0579 | 75.2 | 13.2 | 11.6 | 0.0699 | — |
| Example 15 | 43.5 | 21.1 | 721.0 | 28.3 | 30.2 | 0.7296 | 82.3 | 7.9 | 9.6 | 0.1625 | — |
| Example 16 | 96.8 | 47.0 | 5.4 | 0.1 | 0.2 | 0.0011 | 89.2 | 4.9 | 5.9 | 0.0026 | — |
| Example 17 | 72.5 | 35.1 | 168.0 | 8.2 | 11.3 | 0.0558 | 80.1 | 7.7 | 12.2 | 0.0891 | — |
| Example 18 | 68.1 | 33.9 | 143.0 | 10.1 | 12.5 | 0.1973 | 76.3 | 9.8 | 13.8 | 0.1021 | — |
| Comparative | 32.9 | 16.0 | 54.0 | 2.1 | 4.8 | 0.0693 | 79.9 | 7.3 | 12.8 | 0.3096 | 63.8 |
| Example1 | | | | | | | | | | | |
| Comparative | 34.6 | 16.9 | 63.8 | 3.3 | 5.6 | 0.0779 | 80.3 | 9.3 | 10.4 | 0.4229 | 59.2 |
| Example 2 | | | | | | | | | | | |

DDE: diethyl ether

### INDUSTRIAL APPLICABILITY

It is apparent from the foregoing pages that when a catalyst comprising an inorganic support having supported thereon a salt of a heteropolyacid is used, in producing a lower aliphatic carboxylic acid ester from a lower alcohol and a lower aliphatic carboxylic acid, high initial activity and high space time yield are exhibited, a catalyst life sufficiently long to endure practice in industry is ensured and production of by-product compounds harmful to the catalyst can be greatly prevented. Furthermore, by using the catalyst, the reaction for producing a lower aliphatic carboxylic acid ester from a lower alcohol and a lower aliphatic carboxylic acid can be continuously and stably performed for a long period of time.

1. A catalyst, for use in producing a lower aliphatic carboxylic acid ester, which is used in producing a lower aliphatic carboxylic acid ester from a lower alcohol and a lower aliphatic carboxylic acid in a gas phase, wherein said catalyst comprises an inorganic support having supported thereon at least one heteropolyacid and/or heteropolyacid salt.

2. A catalyst as claimed in claim 1, wherein the inorganic support is at least one member selected from the group consisting of silica, alumina, silica alumina and zeolite.

3. A catalyst as claimed in claim 2, wherein silica is silica gel comprising at least SiO₂ in an amount of 90% by mass or more.

4. A catalyst as claimed in any one of claims 1 to 3, wherein the sum total of the heteropolyacid and/or a salt thereof supported is from 50 to 1,000 g based on 1 liter of the inorganic support before the loading of heteropolyacid and/or heteropolyacid salt.

5. A catalyst as claimed in any one of claims 1 to 4, wherein the heteropolyacid is selected from the group consisting of the following heteropolyacids:

| | |
|---|---|
| tungstosilicic acid | H₄[SiW₁₂O₄₀].xH₂O |
| tungstophosphoric acid | H₃[PW₁₂O₄₀].xH₂O |
| molybdophosphoric acid | H₃[PMo₁₂O₄₀].xH₂O |
| molybdosilicic acid | H₄[SiMo₁₂O₄₀].xH₂O |
| vanadotungstosilicic acid | H₄₊ₙ[SiVₙW₁₂₋ₙO₄₀].xH₂O |
| vanadotungstophosphoric acid | H₃₊ₙ[PVₙW₁₂₋ₙO₄₀].xH₂O |
| vanadomolybdophosphoric acid | H₃₊ₙ[PVₙMo_{12 -n}O₄₀].xH₂O |
| vanadomolybdosilicic acid | H₄₊ₙ[SiVₙMo₁₂₋ₙO₄₀].xH₂O |
| molybdotungstosilicic acid | H₄[SiMoₙW₁₂₋ₙO₄₀].xH₂O |
| molybdotungstophosphoric acid | H₃[PMoₙW₁₂₋ₙO₄₀].xH₂O |

wherein n is an integer of 1 to 11 and x is an integer of 1 or more.

6. A catalyst as claimed in any one of claims 1 to 4, wherein the heteropolyacid salt is selected from the group consisting of lithium, cesium, potassium, sodium, magnesium, barium, copper, gold, gallium and ammonia salts of at least one of the following heteropolyacids:

| | |
|---|---|
| tungstosilicic acid | H₄[SiW₁₂O₄₀].xH₂O |
| tungstophosphoric acid | H₃[PW₁₂O₄₀].xH₂O |
| molybdophosphoric acid | H₃[PMo₁₂O₄₀].xH₂O |
| molybdosilicic acid | H₄[SiMo₁₂O₄₀].xH₂O |
| vanadotungstosilicic acid | H₄₊ₙ[SiVₙW₁₂₋ₙO₄₀].xH₂O |
| vanadotungstophosphoric acid | H₃₊ₙ[PVₙW₁₂₋ₙO₄₀].xH₂O |
| vanadomolybdophosphoric acid | H₃₊ₙ[PVₙMo₁₂₋ₙO₄₀].xH₂O |
| vanadomolybdosilicic acid | H₄₊ₙ[SiVₙMo₁₂₋ₙO₄₀].xH₂O |
| molybdotungstosilicic acid | H₄[SiMoₙW₁₂₋ₙO₄₀].xH₂O |
| molybdotungstophosphoric acid | H₃[PMoₙW₁₂₋ₙO₄₀].xH₂O |

wherein n is an integer of 1 to 11 and x is an integer of 1 or more.

7. A process for producing a catalyst, for use in producing a lower aliphatic carboxylic acid ester, said catalyst being used in producing a lower aliphatic carboxylic acid ester from a lower alcohol and a lower aliphatic carboxylic acid in a gas phase, which process comprises the following first and second steps:
First Step
a step of loading a heteropolyacid on an inorganic support to obtain a heteropolyacid supported catalyst; and
Second Step
a step of loading an element of forming a salt on the heteropolyacid supported catalyst obtained in the first step to produce a catalyst for use in producing a lower aliphatic carboxylic acid ester.

8. A process for producing a catalyst, for use in producing a lower aliphatic carboxylic acid ester, said catalyst being used in producing a lower aliphatic carboxylic acid ester from a lower alcohol and a lower aliphatic carboxylic acid in a gas phase, which process comprises a step of loading a heteropolyacid together with a starting material for the element of forming a salt or loading a previously prepared heteropolyacid salt on an inorganic support.

9. A process for producing a catalyst, for use in producing a lower aliphatic carboxylic acid ester, said catalyst being used in producing a lower aliphatic carboxylic acid ester from a lower alcohol and a lower aliphatic carboxylic acid in a gas phase, which process comprises the following first and second steps:
First Step
a step of loading a starting material for the element of forming a heteropolyacid salt on an inorganic support to obtain a salt-forming component supported support; and
Second Step
a step of loading a heteropolyacid on the salt-forming component supported support obtained in the first step to obtain a catalyst for use in producing a lower aliphatic carboxylic acid ester.

10. A process for producing a catalyst, for use in producing a lower aliphatic carboxylic acid ester, said catalyst being used in producing a lower aliphatic carboxylic acid ester from a lower alcohol and a lower aliphatic carboxylic acid in a gas phase, which process comprises a step of loading a heteropolyacid on an inorganic support.

11. A process for producing a lower aliphatic carboxylic acid ester, comprising reacting a lower alcohol and a lower aliphatic carboxylic acid in a gas phase in the presence of a catalyst as set forth in any one of claims 1 to 6.

12. A process as claimed in claim 11, wherein a lower alcohol and a lower aliphatic carboxylic acid are reacted in a gas phase in the presence of water and a catalyst as set forth in any one of claims 1 to 6.

13. A process as claimed in claim 12, wherein the concentration of water is from 1 to 10 mol % based on the total molar number of the lower aliphatic carboxylic acid and the lower alcohol.

14. A process as claimed in any one of claims 11 to 13, wherein the conversion of the lower alcohol is 70% by mass or more.

15. A process as claimed in any one of claims 11 to 14, wherein the ratio of the lower alcohol to the lower aliphatic carboxylic acid is in the range of lower alcohol: lower aliphatic carboxylic acid=1:10 to 1:1 in terms of the molar ratio of the sum totals of respective components.

16. A process as claimed in any one of claims 11 to 15, wherein the lower alcohol contains at least one of an olefin and a diether corresponding to the dehydrated products of the lower alcohol.

17. A process as claimed in any one of claims 11 to 16, wherein the lower alcohol is selected from the group consisting of methanol, ethanol, n-propanol, isopropanol, n-butanol, sec-butanol, isobutanol, tert-butanol, allyl alcohol and crotyl alcohol.

18. A process as claimed in any one of claims 11 to 17, wherein the lower aliphatic carboxylic acid is selected from the group consisting of formic acid, acetic acid, propionic acid, acrylic acid, methacrylic acid and butyric acid.

19. A process as claimed in any one of claims 11 to 18, wherein the lower alcohol is ethanol and the lower aliphatic carboxylic acid is acetic acid.
